(19) United States
(12) Patent Application Publication
(10) Pub. No.: US 2018/0104096 A1
(43) Pub. Date: Apr. 19, 2018

(54) EXOTHERMIC DEVICE

(71) Applicant: KOWA COMPANY, LTD., Nagoya-shi, Aichi (JP)

(72) Inventors: Akiharu Tsuchiya, Koga-shi, Ibaraki (JP); Hodumi Nonaka, Koga-shi, Ibaraki (JP)

(21) Appl. No.: 15/562,816

(22) PCT Filed: Mar. 31, 2016
(86) PCT No.: PCT/JP2016/060639

§ 371 Date: Sep. 28, 2017

(30) Foreign Application Priority Data
Mar. 31, 2015 (JP) 2015-071953

(51) Int. Cl. A61F 7/03

(52) U.S. Cl. CPC A61F 7/034; A61F 2007/0258

(57) ABSTRACT

An object of the present invention is to provide an exothermic device which is capable of reaching a high temperature upon use, and capable of sustaining a high temperature state for a prolonged period of time. The present invention provides an exothermic device (1) including: an exothermic agent (5); and a packaging material enclosing the exothermic agent (5). The packaging material is in the form of a flat bag composed of a first surface and a second surface. Each of the first surface and the second surface includes a breathable region (2) and a non-breathable region (3).

### TECHNICAL FIELD

The present invention relates to an exothermic device utilizing an exothermic reaction between an oxidizable metal powder and air.

### BACKGROUND ART

An exothermic device which utilizes an exothermic reaction between an oxidizable metal powder and air and which is represented by a disposable body warmer (hereinafter referred to as exothermic device) is a device for providing warmth, and it is preferred for its disposability and convenience of portability.

A common exothermic device includes: an exothermic agent composed of a mixture of an oxidizable metal powder (iron powder), wood chips, vermiculite, a water-absorbing polymer, activated carbon, a salt, water and the like; and a bag-like packaging material made of a resin film, a nonwoven fabric, a paper, and/or the like, and enclosing the exothermic agent. In general, the exothermic device is packaged in a sealed outer package during storage. When the exothermic device is taken out of the sealed outer package upon use, oxygen in the air is taken into the exothermic device from the exterior thereof, through a breathable region(s) of the packaging material, to allow for an oxidation reaction of the exothermic agent. Thus, the heat generated by the reaction can be used for providing warmth.

Many of the commercially available exothermic devices have: a rise time to reach a temperature of 40° C. of about from 10 to 20 minutes; an average temperature during use of around 50° C.; the highest temperature of around 60° C.; and a duration during which a temperature of 40° C. or higher is sustained of about from 8 to 24 hours, as evaluated in accordance with JIS S 4100-2007 (hereinafter, simply referred to as JIS S 4100).

Various investigations on the form of packaging materials have been done so far, in order to adjust the rising of heat, the exothermic temperature (temperature of generated heat), the duration during which the generated heat is sustained, and the like of exothermic devices.

For example, Patent Document 1 discloses that it was possible to shorten the rise time for generating heat, by providing breathable regions, divided in right and left regions, on one surface of the packaging material of an exothermic device. Further, Patent Document 2 discloses that it is possible to adjust the exothermic temperature and the duration during which the generated heat is sustained, by providing a sheet capable of blocking air, on the surfaces of the packaging material of an exothermic device whose entire surfaces consist of breathable regions, and by arbitrarily peeling off the sheet to adjust the area of the breathable region.

### PRIOR ART DOCUMENTS

### Patent Documents

Patent Document 1: Utility Model Registration No. 3153612

Patent Document 2: Utility Model Registration No. 3140728

### SUMMARY OF THE INVENTION

### Problems to be Solved by the Invention

There is a tendency that exothermic devices capable of reaching a higher temperature are preferred, in cases where the exothermic devices are used for outdoor sports or work activities in winter, or used in cold regions. Further, there is also a demand for exothermic devices capable of providing warmth for a prolonged period of time.

However, although exothermic devices currently available in the market as high temperature type devices are capable of reaching the highest temperature of 60° C. or higher, the period of time for which these devices can sustain a high temperature state is not so long. In addition, there are various examples of exothermic devices in which the forms of the packaging materials are devised, as described above, but none of them have achieved reaching a high temperature and sustaining a high temperature state at the same time. When the surrounding temperature is low, in particular, the exothermic performance of an exothermic device is decreased accordingly. Therefore, for practical use in a low temperature environment, an exothermic device is required which exhibits capabilities to reach a high temperature and to sustain a high temperature state, in the evaluation in accordance with JIS S 4100. However, no satisfactory exothermic device has been proposed so far.

In view of such a situation, an object of the present invention is to provide an exothermic device which is capable of reaching a high temperature upon use, and capable of sustaining a high temperature state for a prolonged period of time. In particular, an object of the present invention is to provide an exothermic device which is capable of providing a sufficient feeling of warmth for a prolonged period of time, even under a low temperature environment.

### Means for Solving the Problems

As a result of intensive studies in order to solve the above mentioned problems, the present inventor has found out that it is possible to provide an exothermic device which is capable of both reaching a high temperature and sustaining a high temperature state at the same time, by providing a breathable region and a non-breathable region in each of both surfaces of the exothermic device having a flat shape, thereby completing the present invention.

In other words, the present invention is as follows.

[1] An exothermic device including: an exothermic agent; and a packaging material enclosing the exothermic agent,

wherein the packaging material is in the form of a flat bag composed of a first surface and a second surface, and

wherein each of the first surface and the second surface includes a breathable region and a non-breathable region.

[2] The exothermic device according to [1], wherein each of the first surface and the second surface includes one breathable region.
[3] The exothermic device according to [1] or [2], wherein the breathable region is in the form of a belt extending in the longitudinal direction or the transverse direction of the packaging material.
[4] The exothermic device according to any one of [1] to [3], wherein the non-breathable region in the second surface is located at a portion thereof opposed to the breathable region in the first surface, and wherein the non-breathable region in the first surface is located at a portion thereof opposed to the breathable region in the second surface.
[5] The exothermic device according to [4], wherein the breathable region in the first surface and the breathable region in the second surface are located symmetrically with respect to the center line along the longitudinal direction or the transverse direction of the packaging material.
[6] The exothermic device according to any one of [1] to [5], wherein the packaging material is obtained by laminating a nonwoven fabric and a resin film, and wherein the breathable region is formed by providing a plurality of small holes penetrating through the packaging material.
[7] An exothermic device including: an exothermic agent; and a packaging material enclosing the exothermic agent,

wherein the packaging material is in the form of a flat bag composed of a first surface and a second surface,

wherein each of the first surface and the second surface includes a breathable region and a non-breathable region, and

wherein the breathable region in the first surface and the breathable region in the second surface are provided continuously over both surfaces across a long side or a short side of the packaging material.

[8] The exothermic device according to [7], wherein the breathable region is in the form of a belt extending in the longitudinal direction or the transverse direction of the packaging material.
[9] The exothermic device according to [7] or [8], wherein each of the first surface and the second surface includes two breathable regions.

### Effect of the Invention

The present invention provides an exothermic device which is capable of reaching a high temperature upon use, and capable of sustaining a high temperature state for a prolonged period of time. Such an exothermic device is suitable for use in low temperature environments, such as outdoors in winter and cold regions, or for users who prefer a higher temperature.

### BRIEF DESCRIPTION OF THE DRAWINGS

FIG. 1(a) and FIG. 1(b) are diagrams showing one embodiment of an exothermic device according to the present invention. The line A-B indicates the center line along the transverse direction, and the line C-D indicates the center line along the longitudinal direction. FIG. 1(a) shows the appearance of a first surface, and FIG. 1(b) shows the appearance of a second surface, of the exothermic device.

FIG. 2(a) to FIG. 2(e) are diagrams showing cross sections of exothermic devices in the transverse direction. Adhesive portions are not shown in the figures. FIG. 2(a) shows a sectional view of the exothermic device shown in FIG. 1, taken along the line A-B. Further, the exothermic devices shown in FIG. 2(a) to FIG. 2(d) correspond to the exothermic devices produced in Examples 1 to 4, respectively, and the exothermic device shown in FIG. 2(e) corresponds to the exothermic device produced in Comparative Example 1. The point E indicates the position of the center line along the longitudinal direction.

FIG. 3(a) and FIG. 3(b) are diagrams showing one embodiment of the exothermic device according to the present invention. The line A-B indicates the center line along the transverse direction, and the line C-D indicates the center line along the longitudinal direction. FIG. 3(a) shows the appearance of the first surface, and FIG. 3(b) shows the appearance of the second surface, of the exothermic device.

FIG. 4(a) and FIG. 4(b) are diagrams showing cross sections of exothermic devices in the transverse direction. The adhesive portions are not shown in the figures. FIG. 4(a) shows a sectional view of the exothermic device shown in FIG. 3, taken along the line A-B. Further, the exothermic device shown in FIG. 4(a) corresponds to the exothermic device produced in Example 5, and the exothermic device shown in FIG. 4(b) corresponds to each of the exothermic devices produced in Comparative Examples 2 and 3. The point E indicates the position of the center line along the longitudinal direction.

FIG. 5 is a graph showing the temperature characteristics of the exothermic devices of Examples 1 and 2.

FIG. 6 is a graph showing the temperature characteristics of the exothermic devices of Examples 1, 3 and 4, and Comparative Example 1.

FIG. 7 is a graph showing the temperature characteristics of the exothermic devices of Example 5, and Comparative Examples 2 and 3.

FIG. 8 is a graph showing the temperature characteristics of the exothermic devices of Examples 1 and 4, and Comparative Example 1, under a low temperature environment (0° C.).

### MODE FOR CARRYING OUT THE INVENTION

The exothermic device according to the present invention includes: an exothermic agent; and a packaging material enclosing the exothermic agent, and the exothermic device can be used as a disposable body warmer, for providing warmth to a human body or other objects.

The packaging material in the present invention is in the form of a flat bag composed of a first surface and a second surface. The first surface and the second surface as used herein refer to a substantially flat surface on one side, and to a substantially flat surface on the other side, respectively, of the packaging material which is in the form of a flat bag. The present invention includes both the cases in which the first surface and the second surface are formed of the same (a continuous) member of the packaging material, and in which the first surface and the second surface are formed of a plurality of members of the packaging material.

The shape of the exothermic device according to the present invention is not particularly limited. However, the exothermic device usually has a substantially rectangular shape. The size of the exothermic device is not particularly limited. However, the exothermic device preferably has, for example, a length in the longitudinal direction of from 50 to 150 mm, a length in the transverse direction of from 40 to 140 mm, and a thickness of from 2 to 15 mm when the exothermic agent is evenly distributed within the packaging material, because having such a size allows the exothermic device to be easily held in a hand or placed in a pocket.

Each of the first surface and the second surface of the packaging material in the present invention includes a breathable region and a non-breathable region. Each of the first surface and the second surface includes one or more of the breathable regions. However, it is preferred that each surface include one breathable region.

The ratio of the total area of the breathable region(s) to the total area of the non-breathable region(s) in each surface is not particularly limited. However, the ratio is preferably within the range of from 1:5 to 1:1, and more preferably from 1:4 to 1:2. When the ratio is adjusted within the above described range, it allows the resulting exothermic device to more easily reach a high temperature state, and to more easily sustain the high temperature state.

The breathable region as used herein refers to a region which allows the passage of air therethrough from the exterior to the interior of the packaging material. The non-breathable region as used herein refers to a region which blocks the passage of air between the exterior and the interior of the packaging material.

In general, the breathable region is formed by providing a plurality of small holes penetrating through the packaging material. The size of the small holes is not particularly limited as long as it is sufficient for blocking the exothermic agent inside the packaging material to pass therethrough. For example, the small holes have a diameter of from 0.01 to 0.5 mm. The shape of the small holes is not particularly limited, as well.

The breathable region can be formed, for example, by: providing the small holes linearly along the longitudinal direction or the transverse direction of the packaging material at regular intervals, to form a line of small holes; and providing the lines of small holes at regular intervals in a number of from one to 15 lines, preferably from three to 15 lines, and more preferably from six to 15 lines.

The interval between two adjacent small holes in one line of small holes is not particularly limited. However, the interval is preferably from 1 to 6 mm, and more preferably from 2 to 4 mm.

In cases where a plurality of lines of small holes is provided, the interval between two adjacent lines of small holes is not particularly limited. However, the interval is preferably from 1 to 6 mm, and more preferably from 2 to 4 mm. In this case, the width of the breathable region is not particularly limited, because the width may be adjusted as appropriate depending on the size of the packaging material to be used. However, the width is preferably from 5 to 50 mm, more preferably from 10 to 40 mm, and still more preferably from 15 to 30 mm.

Further, the length of one line of small holes is arbitrary, and the lengths of the plurality of lines of small holes may be the same as or different from each other.

The breathable region preferably has an air permeability of from 11 to 15 s/800 cc·52 cm², since it allows the resulting exothermic device to more easily reach a high temperature state, but not particularly limited thereto. The air permeability as used herein is a value measured in accordance with the Gurley test method (JIS P8117), except for changing the area to be measured to 52 cm².

Preferred embodiments of the exothermic device according to the present invention will now be described, with reference to drawings.

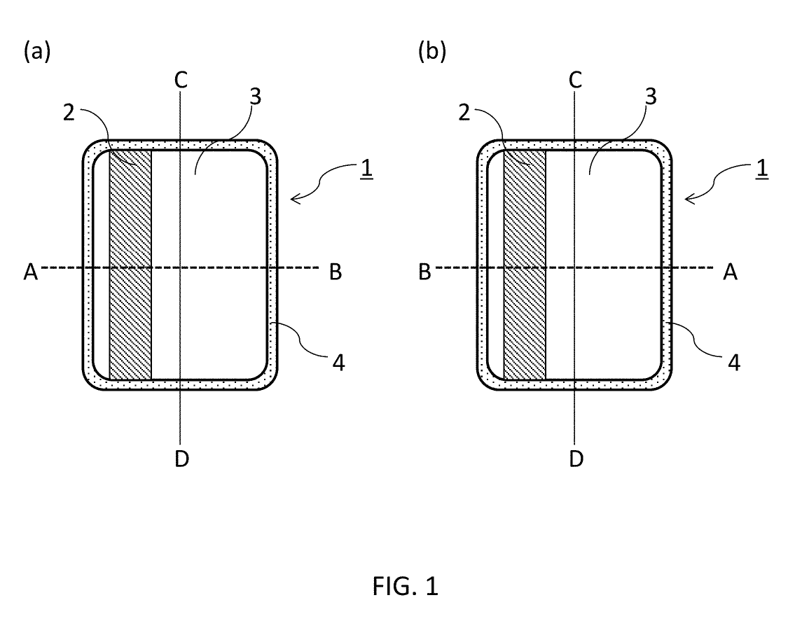

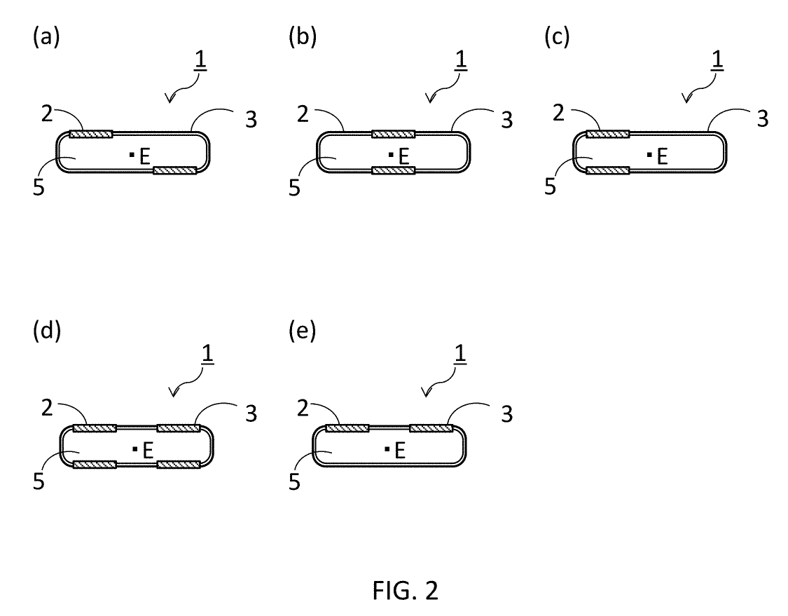

FIG. 1(a) and FIG. 1(b) show one embodiment of an exothermic device 1 according to the present invention. FIG. 1(a) is a plan view showing the appearance of the first surface, and FIG. 1(b) is a plan view showing the appearance of the second surface. Two pieces of packaging materials having a substantially rectangular shape were adhered to each other via adhesive portions 4 provided at the outer peripheral portions of the respective pieces of packaging materials, to form a bag-like packaging material, with the exothermic agent enclosed inside the resulting bag-like packaging material. Thus, an exothermic device having a flat shape is formed. Each of the first surface and the second surface includes one breathable region 2, and the breathable region 2, which is in the form of a belt and extending in parallel with center line CD along the longitudinal direction, extends from one short side of the packaging material to the other short side thereof. The belt-like breathable region 2 in each surface is preferably located at a position away from the center line CD along the longitudinal direction, toward one long side of the packaging material. It is more preferred that the ratio of the distance (interval) from the center line CD along the longitudinal direction to the center line of the breathable region 2, to the distance (interval) from the center line of the breathable region 2 to the long side of the packaging material on the side closer to the breathable region 2, be within the range of from 1:1 to 5:1. Further, each of the first surface and the second surface includes a non-breathable region 3. FIG. 2(a) shows a sectional view of the exothermic device shown in FIG. 1, taken along the line A-B. The non-breathable region 3 in the second surface is located at a portion thereof opposed to the breathable region 2 in the first surface, and the non-breathable region 3 in the first surface is located at a portion thereof opposed to the breathable region 2 in the second surface. It is more preferred that the breathable region 2 in the first surface and the breathable region 2 in the second surface are located symmetrically with respect to the center line CD along the longitudinal direction of the packaging material. In the present specification, the expression “located symmetrically with respect to the center line CD along the longitudinal direction of the packaging material” means that the breathable region 2 in the first surface and the breathable region 2 in the second surface are arranged such that, when the exothermic device is rotated 180 degrees about the center line CD along the longitudinal direction of the packaging material, the location of the breathable region 2 in the first surface after the rotation partially or entirely overlaps with the location of the breathable region 2 in the second surface before the rotation.

When the breathable regions are arranged as described above, the oxidation of the exothermic agent is facilitated to allow the resulting exothermic device to more easily reach a high temperature state, and at the same time, an excessive exothermic reaction is inhibited to allow the exothermic device to more easily sustain the high temperature state.

In the exothermic device according to the present invention, the breathable region 2 in the second surface may be located opposite to the breathable region 2 in the first surface, as shown in FIG. 2(b) and FIG. 2(c), as long as each of the first surface and the second surface of includes a breathable region and a non-breathable region. Further, the present invention also includes an embodiment in which each of the first surface and the second surface includes two breathable regions 2, as shown in FIG. 2(d).

Further, the present invention also includes a case in which each surface of the exothermic device includes a belt-like breathable region extending in parallel with the center line along the transverse direction.

In this case, the breathable region in each surface is preferably located at a position away from the center line along the transverse direction, toward one short side of the packaging material. It is more preferred that the ratio of the distance (interval) from the center line along the transverse direction to the center line of the breathable region, to the distance (interval) from the center line of the breathable region to the short side of the packaging material on the side closer to the breathable region be within the range of from 1:1 to 6:1. Further, it is preferred that the non-breathable region in the second surface be located at a portion thereof opposed to the breathable region in the first surface, and the non-breathable region in the first surface be located at a portion thereof opposed to the breathable region in the second surface. It is more preferred that the breathable region in the first surface and the breathable region in the second surface be located symmetrically with respect to the center line along the transverse direction of the packaging material. In the present specification, the expression “located symmetrically with respect to the center line along the transverse direction of the packaging material” means that the breathable region in the first surface and the breathable region in the second surface are arranged such that, when the exothermic device is rotated 180 degrees about the center line along the transverse direction of the packaging material, the location of the breathable region in the first surface after the rotation partially or entirely overlaps with the location of the breathable region in the second surface before the rotation.

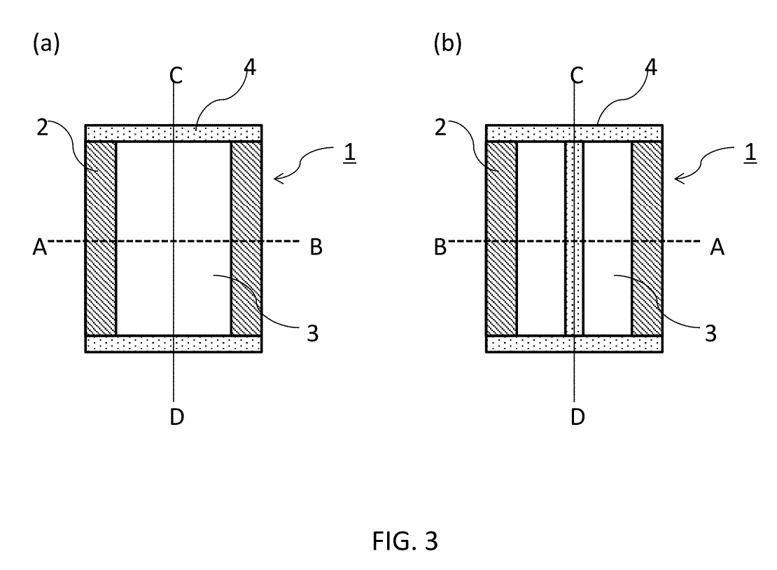

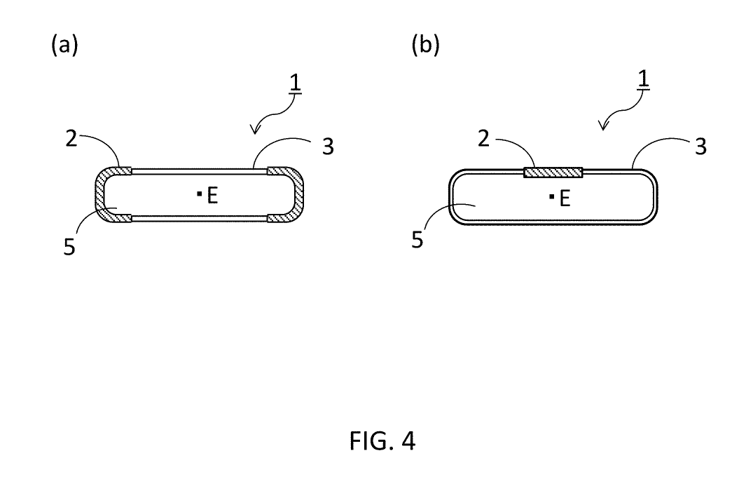

FIG. 3(a) and FIG. 3(b) show another embodiment of the exothermic device 1 according to the present invention. FIG. 3(a) is a plan view showing the appearance of the first surface, and FIG. 3(b) is a plan view showing the appearance of the second surface. The packaging material which is in the form of a substantially rectangular bag includes adhesive portions 4 one each at two short side portions. Further, a belt-like adhesive portion 4 extending in the longitudinal direction is provided at the inner side of the second surface. The exothermic agent is enclosed inside the bag-like packaging material, to form an exothermic device having a flat shape. Preferably, adhesive portions are not provided at the long side portions of the packaging material. Each of the first surface and the second surface includes the breathable regions 2 and the non-breathable region 3. The breathable regions 2, which are in the form of a belt and extending in parallel with the center line CD along the longitudinal direction, extend from one short side of the packaging material to the other short side thereof, and are provided continuously over the first surface and the second surface across both long sides (see the sectional view shown in FIG. 4(a)). In other words, the breathable regions 2 in the first surface and the breathable regions 2 in the second surface are provided continuously. In the exothermic devices shown in FIG. 3 and FIG. 4(a), each of the first surface and the second surface includes two breathable regions 2; however, each surface may include one breathable region 2.

The embodiment of the present invention shown in FIG. 3 can be suitably used in the case of producing a small-size exothermic device having a better portability. For example, this embodiment is preferably used in an exothermic device having a length in the longitudinal direction of from 50 to 120 mm, a length in the transverse direction of from 40 to 70 mm, and a thickness of from 2 to 15 mm when the exothermic agent is evenly distributed within the packaging material.

In such a small-size exothermic device, the adhesive portions are preferably not provided at the long side portions of the packaging material, as described above. Further, the adhesive portion extending in the longitudinal direction is preferably provided at the inner side of the second surface, and more preferably at the center of the second surface. This arrangement allows for providing a favorable feeling upon use, because a rigid feeling due to the adhesive portions of the packaging material is less likely to be perceived when held in a hand, and the cylinder-like shape of the packaging material fits well to the hand.

When producing an exothermic device according to this embodiment, the exothermic device can be produced, for example, by a method in which: one piece of sheet which serves as the material for the packaging material is cut out in the shape of a rectangle; two opposing sides of the sheet are adhered with each other, so that the sheet is formed in the shape of a cylinder; and then each of the upper end and the lower end of the cylinder is closed by adhesion, with the exothermic agent enclosed inside the cylinder-like packaging material.

In the exothermic device according to the present invention, the highest temperature as measured in accordance with JIS S 4100 is preferably from 65 to 80° C., more preferably from 67 to 75° C., and still more preferably from 69 to 72° C., for example. Further, a high temperature state of 65° C. or higher is more easily sustained. For example, the high temperature state is sustained preferably for two hours or more, more preferably four hours or more, and still more preferably six hours or more. Still further, the average temperature as measured in accordance with JIS S 4100 is preferably 55° C. or higher, more preferably 60° C. or higher, and still more preferably 62.5° C. or higher.

The packaging material in the exothermic device according to the present invention is not particularly limited, and any packaging material used in a conventional disposable body warmer or the like can be used. Preferred examples thereof include a sheet material obtained by laminating a nonwoven fabric and a resin film, which sheet is usually used with the side of the nonwoven fabric being the outer side. Further, the sheet material may include an intermediate layer between the nonwoven fabric and the resin film. The nonwoven fabric and the resin film may be laminated with each other with an adhesive layer, such as one made of a hot-melt type adhesive, interposed therebetween.

Examples of the material which can be used for producing the nonwoven fabric include known or commonly used nonwoven fabrics (nonwoven fabrics made of natural fibers, and nonwoven fabrics made of synthetic fibers), for example: nonwoven fabrics made of polyesters (such as nonwoven fabrics made of polyethylene terephthalate); nonwoven fabrics made of polyolefins (such as nonwoven fabrics made of polyethylene, and nonwoven fabrics made of polypropylene); nonwoven fabrics made of polyamides (such as nonwoven fabrics made of Nylon); and nonwoven fabrics made of rayon; but not particularly limited thereto.

Further, examples of the method of producing the nonwoven fabric include: a spun-bond method, a spun-lace method, a melt blow method, a thermal bond method, a chemical bond method, a needle punch method, and the like, but not particularly limited thereto.

The nonwoven fabric preferably has a basis weight of from 20 to 40 g/m², for example.

Examples of the material which can be used for producing the resin film include: ethylene resins such as low density polyethylenes, straight-chain low density polyethylenes, medium density polyethylenes, high density polyethylenes, ethylene-vinyl acetate copolymers, and ethylene-α-olefin copolymers; propylene resins such as polypropylenes, and propylene-α-olefin copolymers; polybutene resins such as polybutene-1; ethylene-unsaturated carboxylic acid copolymers such as poly-4-methylpentene-1, ethylene-acrylic acid copolymers, and ethylene-methacrylic acid copolymers; ethylene-(meth)acrylate copolymers such as ethylene-methyl acrylate copolymers, ethylene-ethyl acrylate copolymers, and ethylene-methyl methacrylate copolymers; ethylene-vinyl alcohol copolymers; and the like, but not particularly limited thereto.

The resin film preferably has a density of from 0.90 to 0.93 g/cm³, for example.

Examples of the material for the intermediate layer include other nonwoven fabrics, other resin films, titanium white, aluminum, and the like, but not particularly limited thereto.

The exothermic device according to the present invention can be formed with or without a tackifier portion(s) provided in the non-breathable region(s) of the packaging material, to be used as a stick-on type which can be pasted on clothes or on the skin, or as a non-stick-on type. However, it is preferred that the exothermic device be formed without the tackifier portion(s), and used as a non-stick-on type.

The exothermic agent in the exothermic device according to the present invention is not particularly limited, and any exothermic body used in a conventional disposable body warmer can be used. For example, an oxidizable metal powder such as iron powder, activated carbon, water, a water-holding agent (such as wood flour, vermiculite, diatomaceous earth, perlite, silica gel, alumina, and/or a water-absorbing polymer), an inorganic salt (such as sodium chloride or potassium chloride), and the like may be enclosed in the packaging material, in an adequate amount depending on the capacity of the packaging material.

As an example, a mixture obtained by mixing the following ingredients at the following composition can be used as the exothermic agent: from 50 to 60 parts by mass of iron powder; from 3 to 10 parts by mass of activated carbon; from 2 to 5 parts by mass of sodium chloride; from 5 to 20 parts by mass of water-holding material; and from 20 to 32 parts by mass of water.

### EXAMPLES

The present invention will now be described in detail with reference to Examples. However, the present invention is in no way limited by the following Examples.

### Production Example 1 of Exothermic Device

Two pieces of sheets, each obtained by laminating a Nylon nonwoven fabric, a polyethylene film and a straight chain low density polyethylene film in this order, were layered one on another, with the sides of the Nylon nonwoven fabrics being the outer sides. Then the outer peripheries of the layered sheets were adhered with each other by thermocompression bonding, with 51 g of exothermic agent enclosed therebetween, to produce an exothermic device having the shape of a substantially rectangular flat bag and having a size of 132 mm×100 mm. The components of the exothermic agent are iron powder, activated carbon, sodium chloride, a water-holding material, and water. In each of the sheets, needle-punched holes each having a diameter of 0.05 mm were provided at intervals of 2 mm to form a line of needle-punched holes, and eleven of the lines of needle-punched holes were provided at intervals of 2 mm, to form a belt-like breathable region extending in the longitudinal direction and having an air permeability of 12.5 s/800 cc·52 cm². Specifically, the following five types of exothermic devices were produced. An exothermic device in which: the breathable regions are provided one each at both surfaces; the breathable region in the first surface and the breathable region in the second surface are arranged symmetrically with respect to the center line along the longitudinal direction of the packaging material; and the breathable region in each surface is provided in a width of 20 mm, from the position 15 mm away from the center line, toward the long side of the packaging material, on the side provided with the breathable region (Example 1: FIG. 2 (a)). An exothermic device in which: the breathable regions are provided one each at both surfaces; and the breathable region in the first surface and the breathable region in the second surface are each provided in a width of 20 mm at the center of each surface, along the longitudinal direction of the packaging material (Example 2: FIG. 2 (b)). An exothermic device in which: the breathable regions are provided one each at both surfaces; and the breathable region in the first surface and the breathable region in the second surface are provided in a width of 20 mm, from the positions 15 mm away from the center line along the longitudinal direction of the packaging material, on the same side with respect to the center line, toward the same long side of the packaging material (Example 3: FIG. 2 (c)). An exothermic device in which: the breathable regions are provided two each at both surfaces; and the breathable regions in the first surface and the breathable regions in the second surface are provided in a width of 20 mm, from the positions 15 mm away from the center line along the longitudinal direction of the packaging material, respectively toward both long sides of the packaging material (Example 4: FIG. 2 (d)). An exothermic device in which: two breathable regions are provided on only one surface; and the breathable regions are provided in a width of 20 mm, from the positions 15 mm away from the center line along the longitudinal direction of the packaging material, respectively toward both long sides of the packaging material (Comparative Example 1: FIG. 2 (e)).

### Production Example 2 of Exothermic Device

The same sheet as used in the above described Production Example 1 was cut out in the shape of a rectangle, and two opposing sides of the sheet were adhered with each other by thermocompression bonding, such that the sheet is formed in the shape of a cylinder, with the side of the Nylon nonwoven fabric being the outer side. Then each of the upper end and the lower end of the cylinder was closed by thermocompression bonding, with 19 g of exothermic agent (the same as that used in Production Example 1) enclosed in the cylinder, to produce an exothermic device having the shape of a substantially rectangular flat bag and having a size of 93 mm×55 mm. Specifically, the following three types of exothermic devices were produced. An exothermic device in which: two breathable regions are provided; and the breathable regions are in the shape of a belt extending in the longitudinal direction of the packaging material, and provided in a width of 18 mm, so as to extend continuously over both surfaces across both long sides of the packaging material (Example 5: FIG. 4 (a)). An exothermic device in which: one breathable region is provided on only one surface; and the breathable region is in the shape of a belt extending in the longitudinal direction of the packaging material, and provided in a width of 16 mm at the center of the surface, along the longitudinal direction of the packaging material (Comparative Example 2: FIG. 4 (b)). An exothermic device in which the breathable region is provided in the same manner as in Comparative Example 2, except for changing the width thereof to 24 mm (Comparative Example 3: FIG. 4 (b)). In the above described exothermic devices, needle-punched holes were provided such that the air permeability of the breathable regions in the respective exothermic devices were 13.2 s/800 cc·52 cm² (Example 5), 14.8 s/800 cc·52 cm² (Comparative Example 2), and 12.0 s/800 cc·52 cm² (Comparative Example 3), respectively.

### <Temperature Characteristics Test>

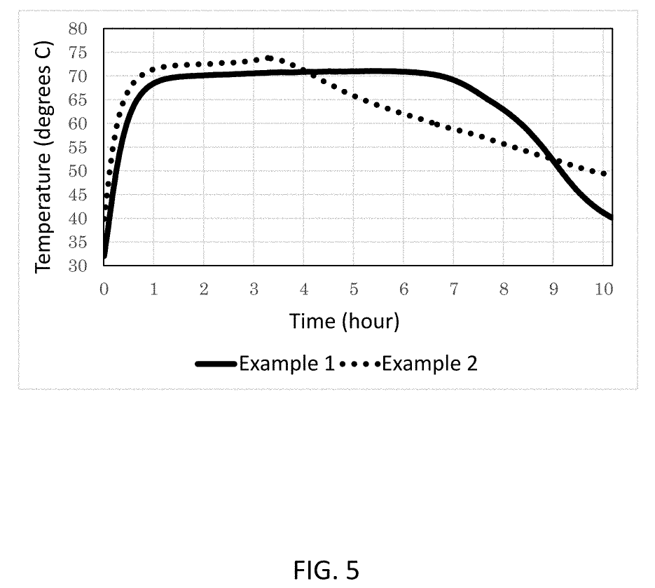

The temperature characteristics of the exothermic devices produced in Production Examples 1 and 2 were tested in accordance with the testing method defined in JIS S 4100. The results are shown in FIG. 5 to FIG. 7.

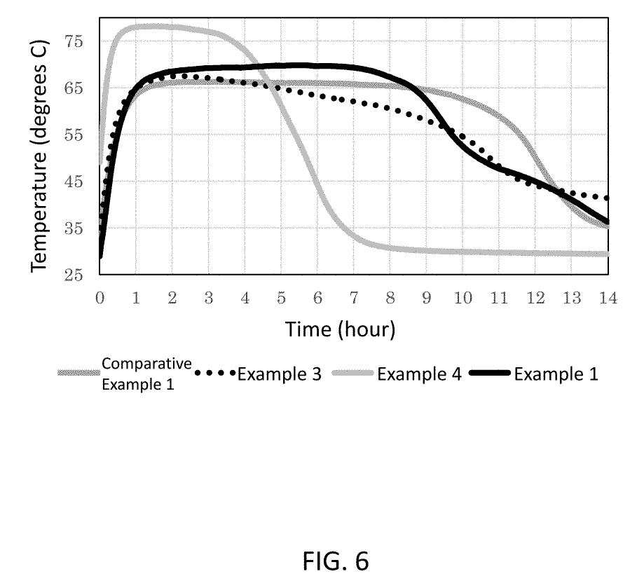

The exothermic device produced in Example 1 reached the highest temperature of about 71° C., sustained a state of 65° C. or higher for about seven hours, and had an average temperature of about 65° C. The exothermic device produced in Example 2 reached the highest temperature of about 73° C., sustained a state of 65° C. or higher for about 4.5 hours, and had an average temperature of about 58° C. (FIG. 5). In another test, the exothermic device produced in Example 1 reached the highest temperature of about 70° C., sustained a state of 65° C. or higher for about eight hours, and had an average temperature of about 61° C. The exothermic device produced in Example 3 reached the highest temperature of about 67° C., sustained a state of 65° C. or higher for about two hours, and had an average temperature of about 56° C. The exothermic device produced in Example 4 reached the highest temperature of about 78° C., sustained a state of 65° C. or higher for about four hours, and had an average temperature of about 69° C. The exothermic device produced in Comparative Example 1 reached the highest temperature of about 66° C., sustained a state of 65° C. or higher for about eight hours, and had an average temperature of about 62° C. (FIG. 6). It can be seen from these test results that breathable regions are preferably provided one each at both surfaces of the exothermic device, and further, that the breathable regions are more preferably arranged symmetrically with respect to the center line along the longitudinal direction, in order to achieve both reaching a high temperature and sustaining a high temperature state at the same time.

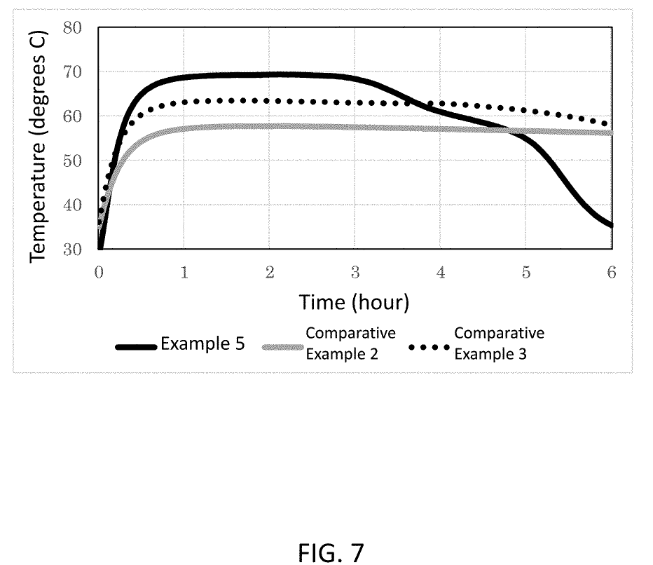

In another test, the exothermic device produced in Example 5 reached the highest temperature of about 70° C., sustained a state of 65° C. or higher for about three hours, and had an average temperature of about 63° C. The exothermic device produced in Comparative Example 2 reached the highest temperature of about 63° C., and the exothermic device produced in Comparative Example 3 reached the highest temperature of about only 58° C., and had an average temperature of 52° C. (FIG. 7). It can be seen from these test results that, the arrangement of breathable regions in which belt-like breathable regions extending in the longitudinal direction are provided so as to extend continuously over both surfaces across both long sides, is also preferred in order to achieve both reaching a high temperature and sustaining a high temperature state at the same time.

Note that, the average temperature as described above was calculated in accordance with the calculation method defined in JIS S 4100.

<Temperature Characteristics Test under Low Temperature Environment (at 0° C.)>

Since there is no method defined by JIS for testing the temperature characteristics under a low temperature environment, the evaluation of the temperature characteristics of the exothermic devices under a low temperature environment was carried out according to the following procedure, with reference to the test method defined in JIS S 4100.

### (1) Test Samples

The respective exothermic devices produced in Example 1, Example 4, and Comparative Example 1 were used as test samples (n=3 in each group).

### (2) Incubator

Low temperature incubator CR-14C (Hitachi Ltd.)

### (3) Temperature Data Logger

Ondotori TR-71wf (T&D Corporation)

### (4) Test Environment

The outer package of each of the test samples was opened in an environment at 20° C., and the measurement was immediately carried out, under an environment of 0° C.×humidity without artificial control.

### (5) Test Method

First, eight sheets of cotton towels (yarn count: 20/1, size: about 34×88 cm, weight per one sheet: about 69 g) were placed in layers to serve as a covering material, in an incubator whose internal temperature was controlled to 0° C., and each test sample was placed on top of the towels such that the exothermic composition was evenly distributed therewithin. Then a temperature data logger was fixed at the center of the bottom surface of the test sample with a cellophane tape (in the case of using as the test sample, an exothermic device in which needle-punched holes are provided only on one surface thereof, the exothermic device was placed, with the surface provided with the needle-punched holes being the upper surface).

Next, the covering material was folded such that the upper surface of the thus placed test sample was also covered therewith, and the measurement of the temperature was started. Measured values of the temperature were recorded every one minute.

### (6) Evaluation Methods

The highest temperature, the average temperature, and the duration, as defined in Table 1, were evaluated with reference to JIS S 4100.

It is said that a human perceives a feeling of warmth at a temperature of 40° C. or higher, and JIS S 4100 defines the duration and the average temperature based on 40° C. In the test performed here under a low temperature environment, as well, the period of time during which the feeling of warmth is perceived, was evaluated by the duration during which a temperature of 40° C. or higher is sustained.

TABLE 1
Evaluated Items and Evaluation Method

| Evaluated | |
|---|---|
| items | Evaluation method |
| Highest | Maximum value of the measured values |
| temperature | |
| (° C.) | |
| Average | Average value of the temperature during the period from |
| temperature | the point at which the temperature reaches 40° C. during |
| (° C.) | its increase, to the point at which the temperature drops |
| | to be 40° C. again during its decrease. |
| Duration (hr) | Period of time from the point at which the temperature |
| | reaches 40° C. during its increase, to the point at which |
| | the temperature drops to be 40° C. again during its |
| | decrease. |

### (7) Results

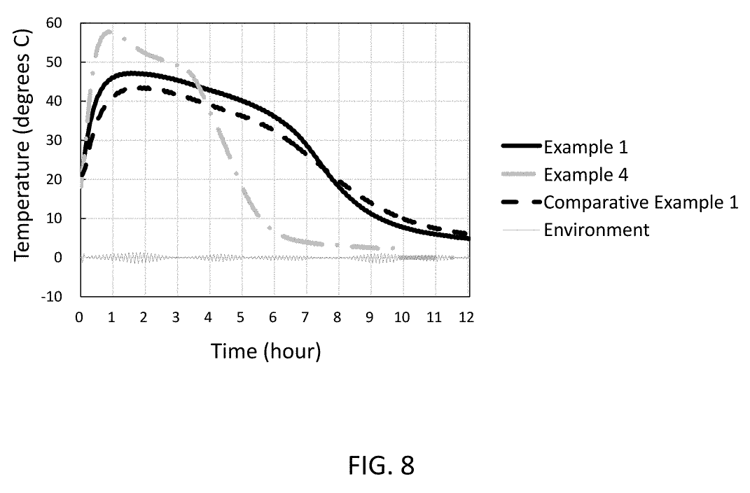

The exothermic devices produced in Example 1, Example 4, and Comparative Example 1 were evaluated under a low temperature environment of 0° C., and the results are shown in FIG. 8 and Table 2. The exothermic device produced in Example 1 reached the highest temperature of 50° C., and sustained a temperature state of 40° C. or higher for 4.5 hours. The exothermic device produced in Example 4 reached the highest temperature of 60.1° C., and sustained a temperature state of 40° C. or higher for 3.8 hours. Further, the exothermic device produced in Comparative Example 1 reached the highest temperature of 45° C., and sustained a temperature state of 40° C. or higher for 2.8 hours.

These test results revealed that, of the exothermic devices of Examples' and 4, and Comparative Example 1, the exothermic device of Example 1 having the longest duration is most preferred, and the exothermic devices of Example 4 is secondly preferred, in order to obtain a sufficient feeling of warmth for a prolonged period of time, even under a low temperature environment. The exothermic device of Comparative Example 1 had a duration which was the shortest, and the highest temperature which was the lowest, of all the samples tested this time.

TABLE 2
Results of Evaluation under Low Temperature Environment

| | | | Comparative |
|---|---|---|---|
| Sample | Example 1 | Example 4 | Example 1 |
| Highest temperature | 50.0 | 60.1 | 45.0 |
| (° C.) | | | |
| Average temperature | 44.6 | 51.2 | 42.2 |
| (° C.) | | | |
| Duration (hr) | 4.5 | 3.8 | 2.8 |

### INDUSTRIAL APPLICABILITY

The present invention serves to provide an exothermic device which is capable of reaching a high temperature upon use, and capable of sustaining a high temperature state for a prolonged period of time. The present invention is industrially useful, since such an exothermic device is suitable for use in outdoor work activities in winter or for use in cold regions.

### DESCRIPTION OF SYMBOLS

1: exothermic device
2: breathable region
3: non-breathable region
4: adhesive portion
5: exothermic agent

1. An exothermic device comprising: an exothermic agent; and a packaging material enclosing the exothermic agent,
wherein the packaging material is in the form of a flat bag composed of a first surface and a second surface, and
wherein each of the first surface and the second surface includes a breathable region and a non-breathable region.

2. The exothermic device according to claim 1, wherein each of the first surface and the second surface includes one breathable region.

3. The exothermic device according to claim 1, wherein the breathable region is in the form of a belt extending in the longitudinal direction or the transverse direction of the packaging material.

4. The exothermic device according to claim 1, wherein the non-breathable region in the second surface is located at a portion thereof opposed to the breathable region in the first surface, and
wherein the non-breathable region in the first surface is located at a portion thereof opposed to the breathable region in the second surface.

5. The exothermic device according to claim 4, wherein the breathable region in the first surface and the breathable region in the second surface are located symmetrically with respect to the center line along the longitudinal direction or the transverse direction of the packaging material.

6. The exothermic device according to claim 1, wherein the packaging material is obtained by laminating a nonwoven fabric and a resin film, and wherein the breathable region is formed by providing a plurality of small holes penetrating through the packaging material.
